(19) United States
(12) Patent Application Publication
(10) Pub. No.: US 2026/0000646 A1
(43) Pub. Date: Jan. 1, 2026

(54) Inhibition of Oral Microbe Biofilms

(71) Applicant: The University of Toledo, Toledo, OH (US)

(72) Inventors: Rudolph E. Sloup, Toledo, OH (US); Jyl S. Matson, Toledo, OH (US)

(73) Assignee: The University of Toledo, Toledo, OH (US)

(21) Appl. No.: 19/252,257

(22) Filed: Jun. 27, 2025

Related U.S. Application Data
(60) Provisional application No. 63/664,947, filed on Jun. 27, 2024.

(51) Int. Cl. A61K 31/41; A23K 20/116; A61K 45/06; A61P 1/02; A61P 31/04

(52) U.S. Cl. CPC A61K 31/41; A23K 20/116; A61K 45/06; A61P 1/02; A61P 31/04

(57) ABSTRACT

Compositions and methods for preventing dental cavities are described. The compositions include, and the methods involve the use of, an anti-biofilm compound such as pranlukast or lithocholic acid.

### RELATED APPLICATIONS

This application claims priority to U.S. Provisional Application No. 63/664,947 filed under 35 U.S.C. § 111(b) on Jun. 27, 2024, the disclosure of which is incorporated herein by reference in its entirety for all purposes.

### STATEMENT REGARDING FEDERALLY SPONSORED RESEARCH

This invention was made with no government support. The government has no rights in this invention.

### BACKGROUND

Streptococcus mutans is a bacterium that lives in the oral cavity of humans and is a major (though not only) cause of caries, also known as dental cavities. In order to cause caries, the bacterium must form a biofilm. The biofilm creates a microenvironment in which the enamel of teeth is degraded and caries form. There is a need for new and improved compositions and methods for preventing dental cavities.

### SUMMARY

Provided is a method of preventing dental cavities in a subject, the method comprising administering an effective amount of an anti-biofilm compound into the mouth of the subject to prevent dental cavities in the subject, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride. In certain embodiments, the subject is a human. In certain embodiments, the subject is an animal with teeth. In particular embodiments, the animal is a dog, a cat, a cow, a chicken, a turkey, a goat, a pig, a sheep, a horse, a fish, a rabbit, a llama, or a deer.

In certain embodiments, the anti-biofilm compound is pranlukast. In certain embodiments, the anti-biofilm compound is lithocholic acid.

In certain embodiments, the TABC is administered through a dental product. In certain embodiments, the TABC is administered through a mouthwash or a toothpaste. In certain embodiments, the TABC is administered through a food product, smokeless tobacco, or candy. In certain embodiments, the effective amount is at least about 10 μM. In certain embodiments, the effective amount is at least about 100 μM. In certain embodiments, the effective amount is at least about 450 μM.

Further provided is a dental composition comprising an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; and one or more dentally acceptable solvents, flavoring agents, preservatives, fragrances, abrasive agents, surfactants, humectants, thickeners, antimicrobial agents, or enamel-strengthening agents. In certain embodiments, the effective amount is at least about 10 μM. In certain embodiments, the effective amount is at least about 100 μM. In certain embodiments, the effective amount is at least about 450 μM. In certain embodiments, the dental composition is a mouthwash, toothpaste, filling material, sealant, coating, cement, or gel.

Further provided is a mouthwash composition comprising an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; and one or more of fluoride, antimicrobial agents, xylitol, alcohol, sodium phosphate, flavoring agents, and water. In certain embodiments, the effective amount is at least about 10 μM. In certain embodiments, the effective amount is at least about 100 μM. In certain embodiments, the effective amount is at least about 450 μM.

Further provided is a dental floss comprising a flexible string comprising nylon, polytetrafluoroethylene (PTFE), polyethylene, silk, or bamboo fiber; and an anti-biofilm compound coated in, on, or impregnated in the flexible string, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride. In certain embodiments, the anti-biofilm compound is part of a flavored or wax coating. In certain embodiments, the anti-biofilm compound is present on or in the dental floss in an amount of at least about 0.0003% w/w. In certain embodiments, the anti-biofilm compound is present on or in the dental floss in an amount in the range of from about 0.0003% w/w to about 5% w/w.

Further provided is a toothpaste composition comprising an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; and one or more abrasive agents, surfactants, humectants, thickeners, and flavoring agents. In certain embodiments, the toothpaste composition further comprises a fluoride compound, a calcium phosphate, or a desensitizing agent. In certain embodiments, the effective amount is at least about 10 μM. In certain embodiments, the effective amount is at least about 100 μM. In certain embodiments, the effective amount is at least about 450 μM.

Further provided is a dental filling material comprising an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; and a base formulation comprising a resinous material. In certain embodiments, the dental filling material further comprises one or more fillers, adhesion promoters, initiators, or modifiers. In certain embodiments, the dental filling material further comprises a colorant, an opacifier, or light-reflective particles. In certain embodiments, the effective amount is at least about 10 μM. In certain embodiments, the effective amount is at least about 100 μM. In certain embodiments, the effective amount is at least about 450 μM.

Further provided is a dental amalgam comprising an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; and a mixture of elemental metals. In certain embodiments, the mixture comprises at least one of silver, tin, copper, and mercury. In certain embodiments, the mixture comprises two or more of silver, tin, copper, and mercury. In certain embodiments, the dental amalgam further comprises an alloying agent. In certain embodiments, the effective amount is at least about 10 μM. In certain embodiments, the effective amount is at least about 100 μM. In certain embodiments, the effective amount is at least about 450 μM.

Further provided is a dental polish comprising an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; a polishing agent; and one or more abrasives, thickeners, humectants, or flavoring agents. In certain embodiments, the dental polish further comprises an enamel-strengthening agent. In certain embodiments, the effective amount is at least about 10 μM. In certain embodiments, the effective amount is at least about 100 μM. In certain embodiments, the effective amount is at least about 450 μM.

Further provided is a dental sealant or coating comprising an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; and a resinous material. In certain embodiments, the dental sealant or coating further comprises one or more cross-linking agents, fillers, adhesion, promoters, or solvents. In certain embodiments, the dental sealant or coating further comprises an enamel-strengthening agent. In certain embodiments, the effective amount is at least about 10 μM. In certain embodiments, the effective amount is at least about 100 μM. In certain embodiments, the effective amount is at least about 450 μM.

Further provided is a chewing gum composition comprising an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; and a gum base, a sweetening agent, and a flavoring agent. In certain embodiments, the sweetening agent comprises a sugar alcohol, an artificial sweetener, or a natural sweetener. In certain embodiments, the chewing gum composition comprises one or more of xylitol, sorbitol, mannitol, aspartame, sucralose, and saccharin. In certain embodiments, the effective amount is at least about 0.0003% w/w. In certain embodiments, the effective amount is in the range of from about 0.0003% w/w to about 5% w/w.

Further provided is a smokeless tobacco composition comprising an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; and tobacco. In certain embodiments, the smokeless tobacco composition further comprises one or more flavorants, sweeteners, or moisture regulators.

Further provided is an animal feed composition comprising one or more base feed ingredients; an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; and optionally, one or more flavor enhancers, binders, excipients, or nutritional supplements.

Further provided is the use of pranlukast or lithocholic acid in a dental product or food item.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: High throughput screen results producing several hits as possible anti-biofilm compounds.

FIG. 2A: Graph showing that pranlukast and lithocholic acid reduce biofilms.

FIG. 2B: Graph showing the same data as FIG. 2A except only for lithocholic acid and pranlukast in comparison to DMSO and negative control.

FIG. 3A: Graph showing that pranlukast and lithocholic acid do not reduce optical density while nifuroxazide does. This is evidence that nifuroxazide kills bacteria while pranlukast and lithocholic acid do not.

FIG. 3B: Graph showing the same data as FIG. 3A except only for lithocholic acid and pranlukast in comparison to DMSO and negative control.

FIGS. 4A-4B: Photographs showing biofilm phenotypes following treatment with, from top to bottom, controls (positive labelled with +, negative labelled with −), oxantel pamoate, amphotericin B, pranlukast, lithocholic acid, and iopanoic acid. From left to right, the wells were treated with 3.16 μM, 10 μM, 31.6 μM, and 100 μM.

FIG. 5A: Graph showing results of analysis of planktonic cells removed from the microtiter plate, as another way of evaluating whether compounds are anti-biofilm compounds. This shows pranlukast and lithocholic acid to be highly effective at 100 μM.

FIG. 5B: Graph showing the same data as FIG. 5A except only for lithocholic acid and pranlukast in comparison to DMSO and negative control.

FIG. 6: Graph showing data reduced example of the true antibiofilm activity of compounds with statistical analysis to determine significance as determined by a T test. *=P<0.001 as compared to DMSO vehicle control.

FIG. 7: Dose response curve showing pranlukast and lithocholic acid efficacy. EC₅₀ values of 24.51 μM for pranlukast and 33.29 μM for lithocholic acid were determined. The EC₅₀ curve was fit using PRISM (Graphpad) with a log-dose vs response, three parameters, nonlinear regression.

FIG. 8: Graph showing that neither pranlukast nor lithocholic acid disperse biofilms. Biofilm dispersal was assayed with treatment of 100 μM compounds. Biofilms were formed for 24 hours then treated for an additional 6 hours. If the compound disperses biofilms the treated black bar should be lower than the light grey in the positive control.

FIGS. 9A-9B: S. mutans was grown in the presence of increasing concentrations of compound and CFUs were plated to assess bacterial survival. FIGS. 9A-9B have lower CFU in the presence of compounds. At no point does the CFU drop to zero indicating that the compounds are not bactericidal, but it is likely they are bacteriostatic.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

True anti-biofilm compounds (TABCs) are substances which inhibit or disperse biofilms without killing bacteria, thereby limiting selective pressure for the development of antibiotic resistance. In accordance with the present disclosure, it has been discovered that certain drugs or other compounds can inhibit Streptococcus mutans from forming the biofilms necessary for the creation of dental cavities. These include oxantel pamoate, amphotericin B, pranlukast, lithocholic acid, iopanoic acid, nitrofural, nifuroxazide, adamantamine fumarate, and telenzepine dihydrochloride. While these compounds may be bacteriostatic, certain of these anti-biofilm compounds may be TABC compounds (as opposed to antibiotics), and all of these anti-biofilm compounds are useful in a wide variety of dental products including, but not limited to, toothpastes, mouth washes, dental floss, dental gels, polishes, sealants, composite fillers, temporary cements, orthodontic bracket cements, oral rinses, denture care products, dental sprays, implants, endodontic sealers or packers, bulk composites, etchants, flowable composites, orthodontic brackets, orthodontic rubber bands, primers, and varnishes, as well as food products such as chewing gum and candy, dog food or other animal feed, and also smokeless tobacco. The anti-biofilm compounds are useful for preventing cavities in humans or animals such as dogs, cats, or other pets, or livestock.

As one example, the drug pranlukast is an antibiofilm compound. Pranlukast, which is also known as N-[4-oxo-2-(1H-tetrazol-5-yl)-4H-chromen-8-yl]-4-(4-phenylbutoxy)benzamide and is commercially available under the brand name Onon, has the following structural formula:

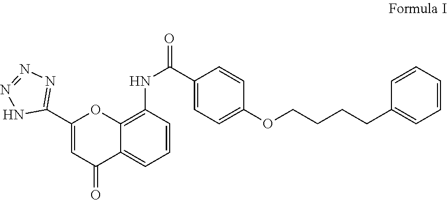 [SMILES: O=C(Nc1cccc2c(=O)cc(-c3nnn[nH]3)oc12)c1ccc(OCCCCc2ccccc2)cc1]

Pranlukast is a cysteinyl leukotriene receptor-1 antagonist used in certain countries for the treatment of allergies. Pranlukast is known for the antagonism of bronchospasm caused by an allergic reaction. However, as demonstrated in the examples herein, pranlukast is useful for the prevention of dental cavities. Pranlukast is commercially available, but may also be prepared, for example, as described in U.S. Pat. No. 5,876,760, which is incorporated herein by reference.

As another example, lithocholic acid is an antibiofilm compound. Lithocholic acid, which is also known as 3α-hydroxy-5β-cholan-24-oic acid or LCA, has the following structural formula:

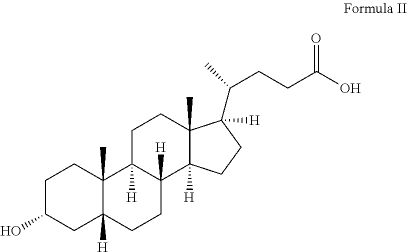

Lithocholic acid is a bile acid which acts as a detergent to solubilize fats for absorption, and also can activate the vitamin D receptor. Lithocholic acid is a secondary bile acid converted in the gut by bacteria. Bacterial action in the colon produces lithocholic acid from chenodeoxycholic acid by reduction of the hydroxyl functional group at carbon-7 in the “B” ring of the steroid framework. Lithocholic acid is also commercially available. As demonstrated in the examples herein, lithocholic acid is useful for the prevention of dental cavities.

Other antibiofilm compounds useful for the prevention of dental cavities include, but are not limited to, oxantel pamoate, amphotericin B, iopanoic acid, nitrofural, nifuroxazide, adamantamine fumarate, and telenzepine dihydrochloride. It should be noted that the exact mechanism of biofilm inhibition/disruption described herein is not known for sure and may be different for different compounds. Without wishing to be bound by theory, it is believed that some of the anti-biofilm compounds act not through dispersal and that the bacteria may not be growing in the presence of the compounds. In any event, the terms “anti-biofouling compound” and “anti-biofilm compound” are used herein to refer to a compound which disrupts, inhibits, disperses, or prevents further growth of bacterial biofilm, without regard for whether the bacteria in question continues to live or grow.

Any, or combinations of, the anti-biofilm compounds described herein can be included in any composition or device designed to be inserted into, or retained within, a mouth, so as to prevent dental cavities. This includes conventional anti-cavity dental products such as mouthwashes and toothpastes, but also includes food or candy items such as chewing gum or suckers, as well as smokeless tobacco, and devices such as retainers, orthodontic rubber bands, biteplates, and palate expanders. In the case of a device, the anti-biofilm compound may be incorporated into a suitable coating on the device which allows for the sustained release of the anti-biofilm compound over time. In the case of a composition such as a mouth wash, the anti-biofilm compound may be incorporated along with other, conventional anti-cavity formulations or the composition may be formulated with the anti-biofilm compound as the main ingredient.

An example mouthwash composition may include an anti-biofilm compound as described herein, such pranlukast and/or lithocholic acid, with one or more mouthwash ingredients such as, but not limited to, fluoride, other antimicrobial agents, xylitol, alcohol, sodium phosphate, flavoring agents, and water. Non-limiting examples of antimicrobial agents include cetylpyridinium chloride (CPC), chlorhexidine, and essential oils such as menthol, eucalyptol, thymol, and methyl salicylate. Xylitol is a sugar alcohol which reduces the growth of bacteria which cause cavities and also helps neutralize acids in the mouth and stimulate saliva production, which can help protect teeth against decay. Alcohol is commonly included in mouthwashes as a solvent and preservative. However, alcohol-free mouthwash compositions are also possible and preferable for some people because alcohol can cause dry mouth or irritation. Sodium phosphate helps remineralize tooth enamel, repairing damage caused by acid erosion and strengthening teeth. In order to improve taste and make the mouthwash more pleasant to use, flavoring agents such as mint, spearmint, peppermint, or fruit flavors may be added. Water is the base ingredient in many mouthwashes, serving as a solvent for other ingredients and helping to distribute them evenly throughout the mouth.

An example toothpaste composition incorporating the anti-biofilm compounds described herein may include an anti-biofilm compound, such as pranlukast and/or lithocholic acid, together with a base formulation containing one or more abrasive agents, surfactants, humectants, thickeners, flavoring agents, and water, which collectively contribute to the cleansing, foaming, viscosity, and sensory attributes of the toothpaste. The anti-biofilm compound is present in an effective concentration, such as about 10 μM or about 100 μM, to exert its anti-caries properties while ensuring compatibility with other formulation components. The toothpaste composition may further include one or more supplementary active ingredients such as fluoride compounds, calcium phosphates, and desensitizing agents, which may enhance the anti-caries efficacy of the toothpaste and promote mineralization of enamel surfaces. These adjunctive ingredients contribute to the overall health benefits of the toothpaste composition, ensuring comprehensive protection against dental caries and maintaining ideal dental hygiene.

An example dental filling material incorporating the anti-biofilm compounds described herein may include an anti-biofilm compound, such as pranlukast and/or lithocholic acid, together with a base formulation composed of resinous materials, fillers, adhesion promoters, initiators, and modifiers. These components facilitate the placement, adaptation, and curing of the filling material within a prepared tooth cavity, ensuring satisfactory physical and mechanical properties of the restoration. Once cured in place in the tooth, the presence of the anti-biofilm compound in the filling material may prevent the formation of cavities on nearby or adjacent tooth surfaces. The dental filling material may further include radiopaque additives, such as barium or strontium compounds, to enable easy identification of the restoration on dental radiographs. Furthermore, the dental filling material may include aesthetic-enhancing ingredients, including colorants, opacifiers, and light-reflective particles. These components ensure that the restoration closely matches the natural shade, translucency, and surface texture of adjacent tooth structure, resulting in seamless integration and superior aesthetics.

An example dental amalgam incorporating the anti-biofilm compounds described herein may include an anti-biofilm compound, such as pranlukast and/or lithocholic acid, together with a mixture of elemental metals, such as silver, tin, copper, and mercury, where the metals are carefully proportioned to achieve the desired mechanical properties, setting characteristics, and biocompatibility of the dental amalgam. The dental amalgam may further include alloying agents, such as indium or palladium, which enhance the physical and mechanical properties of the amalgam, including strength, creep resistance, and corrosion resistance. These alloying agents are added in precise concentrations to achieve desired performance and longevity of the dental restoration.

An example dental polish composition incorporating the anti-biofilm compounds described herein may include an anti-biofilm compound, such as pranlukast and/or lithocholic acid, together with a base composition comprising polishing agents, abrasives, thickeners, humectants, flavoring agents, and water, which collectively facilitate the removal of surface stains, plaque, and biofilm while imparting a pleasant texture and flavor to the polish. The dental polish may further include one or more enamel-strengthening agents, including calcium phosphates, hydroxyapatite nanoparticles, and fluoride compounds, which ingredients penetrate enamel microstructure, replenishing lost minerals and promoting remineralization to combat erosion and surface demineralization. The dental polish composition may further include one or more aesthetic-enhancing ingredients such as polishing silica particles, titanium dioxide, and optical brighteners. These compounds can impart a smooth, lustrous finish to tooth surfaces, masking imperfections and enhancing overall smile aesthetics.

An example dental floss incorporating the anti-biofilm compounds described herein may include a flexible string composed of a nylon, polytetrafluoroethylene (PTFE), polyethylene, silk, or bamboo fiber with the anti-biofilm compound, such as pranlukast and/or lithocholic acid, coated thereon or incorporated thereinto. The anti-biofilm compound may be part of a flavored or wax coating on the dental floss. Other dental flosses are possible and encompassed within the scope of the present disclosure.

As another alternative, the anti-biofilm compounds described herein may be applied to teeth as a sealant or other hardenable coating. Non-limiting example dental sealants or hardenable coatings may include the anti-biofilm compound, such as pranlukast and/or lithocholic acid, together with one or more resinous materials, cross-linking agents, fillers, adhesion promoters, or solvents, which together facilitate the application, adhesion, and curing of the coating onto dental surfaces, ensuring uniform coverage and durability. The sealant or hardenable coating may further include one or more enamel-strengthening agents, such as calcium phosphates, hydroxyapatite nanoparticles, and fluoride compounds, which ingredients penetrate enamel microstructure, replenishing lost minerals and promoting remineralization to fortify tooth structure and reduce susceptibility to damage. The sealant or hardenable coating may further include surface-sealing agents, such as resinous polymers or silanes, which effectively seal microscopic defects and irregularities on dental surfaces. By forming a protective barrier, these agents prevent the ingress of bacteria, acids, and stains, thereby enhancing the longevity and integrity of the dental coating. The sealant or hardenable coating may further include aesthetic-enhancing ingredients, including opacifiers, colorants, and light-reflective particles, which components impart a natural, lustrous appearance to dental surfaces, masking imperfections and enhancing overall smile aesthetics. The sealant or hardenable coating can be applied onto clean and prepared dental surfaces followed by curing under controlled conditions (such as heat or UV light) to achieve sufficient adhesion and hardness. Once cured, the coating forms a resilient and aesthetically pleasing layer that provides long-lasting protection and enhancement onto dental surfaces.

An example animal feed composition incorporating the anti-biofilm compounds described herein may include one or more base feed ingredients, such as grains, vitamins, and minerals suitable for the intended animal species (e.g., corn, soybean meal, wheat bran, fish meal, calcium carbonate, and vitamin supplements); an anti-biofilm compound described herein, such as pranlukast and/or lithocholic acid; one or more flavor enhancers, such as natural or artificial flavors including meat extracts, fruit essences, and savory additives compatible with the dietary preferences of the intended animal species; binders or excipients configured to facilitate the formation and/or processing of the animal feed composition; and optional nutritional supplements, such as amino acids, fatty acids, probiotics, or prebiotics to support overall health and well-being. The anti-biofilm compound may be present in the animal feed composition in an amount of from about 0.0003% by weight to about 20% by weight. The animal feed composition may be tailored for any animal with teeth such as, but not limited to, dogs, cats, cows, chickens, turkeys, goats, pigs, sheep, horses, fish, rabbits, llamas, or deer.

The anti-biofilm compounds described herein, such as pranlukast and/or lithocholic acid, may also simply be added to existing products or formulations such as existing anti-cavity toothpastes and mouthwashes.

The anti-biofilm compounds described herein, such as pranlukast and/or lithocholic acid, can be added to devices or materials used in orthodontics such as braces brackets, rubber bands, cements, and palate expanders. Advantageously, the presence of an anti-biofilm compound in braces may help prevent cavities in areas where a person has difficulty brushing or flossing.

The anti-biofilm compounds may be incorporated within the brackets of braces to provide sustained protection against dental cavities through the orthodontic treatment period. For example, orthodontic brackets can be fabricated using a biocompatible material compatible with the anti-biofilm compound, and the anti-biofilm compound (such as pranlukast and/or lithocholic acid) may be applied or infused within voids or reservoirs within the bracket material during the manufacturing process. The distribution and concentration of the anti-biofilm compound within the brackets can be optimized to ensure maximum efficacy in cavity prevention without compromising the structural integrity or function of the braces.

The anti-biofilm compounds may be incorporated into orthodontic rubber bands, which are commonly used in conjunction with braces to apply force in order to facilitate tooth movement. Rubber bands present challenges regarding oral hygiene and susceptibility to dental decay due to their tight fit and difficulty in cleaning. The integration of an anti-biofilm compound into the rubber bands addresses these concerns by providing a protective barrier against cavity formation. The orthodontic rubber bands can be manufactured using a biocompatible material compatible with the anti-biofilm compound. The anti-biofilm compound, such as pranlukast and/or lithocholic acid, can be incorporated into the orthodontic rubber bands during the manufacturing process either by blending the anti-biofilm compound with the rubber material or by coating the surface of the rubber bands. The distribution and concentration of the anti-biofilm compound within the rubber bands can be optimized to ensure maximum efficacy in cavity prevention without compromising the elasticity or function of the rubber bands.

The anti-biofilm compounds may be incorporated into orthodontic cements by incorporating the anti-biofilm compound, such as pranlukast and/or lithocholic acid, into the cement formulation. Orthodontic cements are used in bonding brackets to teeth, providing stability and facilitating tooth movement during orthodontic treatment. However, cements can contribute to dental decay if proper oral hygiene is not maintained, because the cements create crevices where bacteria can accumulate. The integration of an anti-biofilm compound into an orthodontic cement mitigates this risk by actively preventing cavity formation. The orthodontic cement may be formulated using a biocompatible base material compatible with the anti-biofilm compound, where the anti-biofilm compound is added to the cement in the manufacturing process in a manner which may result in uniform distribution throughout the cement matrix, although uniform distribution throughout the cement matrix is not strictly necessary. The base material of the cement may include a resin or polymer matrix in addition to one or more filler particles, initiators, catalysts, plasticizers, fillers, or pigments. The concentration of the anti-biofilm compound in the cement may be sufficient (such as at least about 10 μM) to provide effective cavity protection without compromising the adhesive properties or biocompatibility of the cement.

As mentioned, the anti-biofilm compounds may also be incorporated into food products such as, but not limited to, chewing gum and candy. An example chewing gum includes an anti-biofilm compound, such as pranlukast and/or lithocholic acid, along with a gum base, a sweetening agent, and a flavoring agent. The gum base may include elastomers, resins, plasticizers, and fillers, which impart chewiness and texture to the chewing gum. Suitable elastomers may include synthetic rubbers such as polyisobutylene, polyethylene, and styrene-butadiene rubber, while suitable resins may include natural resins such as chicle and synthetic resins such as polyvinyl acetate. The sweetening agent may include sugar alcohols, artificial sweeteners, or natural sweeteners. Examples of sugar alcohols include xylitol, sorbitol, and mannitol, while examples of artificial sweeteners include aspartame, sucralose, and saccharin. The flavoring agent may include natural or artificial flavors to impart desirable taste and aroma. Suitable flavoring agents may include mint, fruit flavors, spice extracts, or combinations thereof. The chewing gum can be prepared using conventional techniques, including mixing, extrusion, and shaping processes. The resulting chewing gum product may be packaged in various forms such as sticks, pellets, or tablets for convenient consumption.

The anti-biofilm compound may also be incorporated into smokeless tobacco. An example smokeless tobacco composition may include an anti-biofilm compound, such as pranlukast and/or lithocholic acid, together with tobacco, and optional additives designed to enhance flavor and mouthfeel such as flavorants, sweeteners, or moisture regulators. Example flavorants include natural or artificial tobacco flavors, menthol, spices, and fruit extracts. Example sweeteners include sugar alcohols, artificial sweeteners, or natural sweetening agents. Example moisture regulators include humectants such as glycerin, propylene glycol, and sorbitol, which prevent the tobacco from drying out and ensure a smooth texture. The smokeless tobacco composition may utilize finely ground tobacco flakes, powder, or extract, derived from various tobacco species such as Nicotiana tabacum or Nicotiana rustica. The tobacco may undergo curing, fermentation, and bleeding processes to achieve desired flavor profiles and nicotine content. The tobacco may be contained within a packet and the packet may be made of paper or cloth which may also contain the anti-biofilm compounds. The anti-biofilm compound may be present in the smokeless tobacco composition in an amount ranging from about 0.0003% to about 20% by weight of the total composition, which may ensure the desired dental protection without compromising the sensory attributes of the smokeless tobacco product. The smokeless tobacco product may be prepared using conventional manufacturing techniques, including blending, pressing, and packaging processes. The resulting smokeless tobacco products may be presented in various forms such as loose tobacco, pouches, or chewing tobacco.

In any use or composition, the anti-biofilm compound may be present or may be delivered in an amount effective to inhibit the formation of biofilm. The effective amount may be a concentration of at least about 10 μM. However, in certain embodiments, dental or food compositions may comprise, for example, at least about 0.0003% w/w of an anti-biofilm compound. Furthermore, though the various products may be described herein as including an anti-biofilm compound, it is understood that any such product may include a combination of anti-biofilm compounds, such as including both pranlukast and lithocholic acid.

The anti-biofilm compounds may also be used to prevent biofilm formation on any device or substrate by coating the device or substrate with a coating or film containing the anti-biofilm compound (such as pranlukast and/or lithocholic acid) or otherwise contacting the device or substrate with the anti-biofilm compound. The presence of the anti-biofilm compound will prevent the formation of biofilm from at least Streptococcus mutans on the device or substrate.

The anti-biofilm compounds described herein are thus useful to prevent cavities in a wide variety of products. The anti-biofilm compounds have the advantages of needing only low concentrations, causing less selective pressure for evolution because of a reduced risk of bacterial resistance, and having possible synergies with antibiotics.

### EXAMPLES

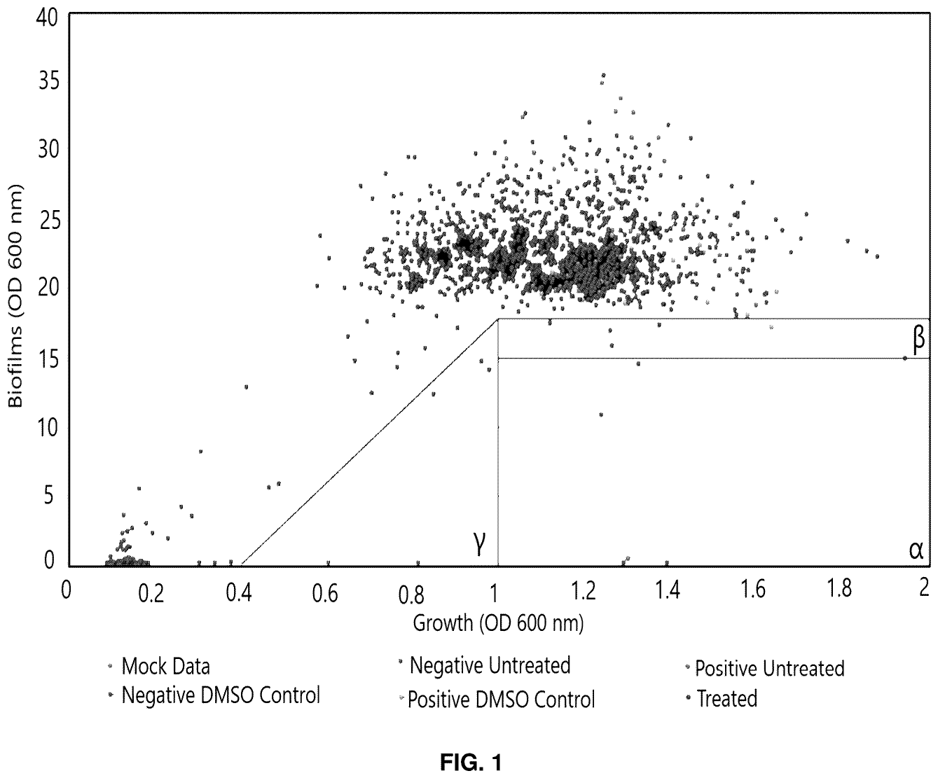

A high throughput screen was used to discover anti-biofilm compounds effective against Streptococcus mutans. The optimal conditions were determined to be cell culture treated 96 well plates, growth overnight at 37° C. ambient atmosphere with 5% CO₂, and Todd Hewitt broth with 5% yeast extract supplemented with 20 mM glucose and 5 mM sucrose. The power of the high throughput screen was validated by determining a Z-factor of 0.76, indicating a robust assay. A library of 1280 compounds was then screened for the ability to inhibit biofilm production, but not inhibit bacterial growth. (FIG. 1.) This yielded several candidates, namely: oxantel pamoate, amphotericin B, pranlukast, lithocholic acid, iopanoic acid, nitrofural, nifuroxazide, adamantamine fumarate, and telenzepine dihydrochloride. Two compounds, pranlukast and lithocholic acid, were verified to have strong biofilm inhibition. At 100 μM, pranlukast inhibited biofilms by 93% and increased the OD of planktonic bacteria by 4.2-fold, while lithocholic acid inhibited biofilms by 91% and increased the planktonic OD by 2.70-fold. Neither compound had the ability to disperse preformed biofilms significantly.

### Materials and Methods

For the biofilm assay screen, the following procedures were followed. S. mutans was cultured overnight in 5 mL of Todd Hewitt broth (BD biosciences) supplemented with 5% yeast extract (BD biosciences) (THY) in a falcon tube at 37° C. and 5% CO₂ from a −80° C. freezer stock. The following day a biofilm assay was prepared using Cyto One tissue culture treated 96 well plates (USA Scientific Inc. Ocala, FL USA) with 200 μL of S. mutans culture per well. The culture was prepared by diluting the overnight culture 1:1000 in THY supplemented with 20 mM glucose and 5 mM sucrose (THYGS) for all biofilm positive wells. Negative biofilm controls were prepared by omitting S. mutans from the well. The microtiter plate was then incubated over night for 20 hours at 37° C. and 5% CO₂ with the sides wrapped in parafilm to reduce evaporation. The next day the biofilms were quantified, and all spectrophotometric reads were measured on a FLUOstar Omega 1300 Series A2 microplate reader (BMG Labtech, Cary, NC USA) at 600 nM optical density. Prior to processing the biofilms, the OD 600 of the plate was read to determine cell growth. Following this, 100 μL of supernatant was pipetted up and down 3 times and then 100 μL of supernatant was carefully removed from each well and placed in a new microtiter plate to quantify any dispersion to the planktonic state. This plate was also measured in the plate reader. Then the remaining planktonic cells were removed from the 96 well plate by inverting it and tapping them into the biohazard waste. Subsequently, 300 μL of phosphate-buffered saline (PBS) was placed in each well two more times and tapped out as above. Then the biofilms were fixed with 300 μL of 200 proof ethanol and incubated at room temperature for 10 minutes. The ethanol was then tapped out and 300 μL of crystal violet (0.41% crystal violet, 12% ethanol) was pipetted into the wells to stain the biofilms and incubated for 10 minutes at room temperature. The crystal violet was then tapped out as above. Next, excess crystal violet was washed out of the wells with PBS by overfilling the entire plate with a serological pipette then tapping the liquid out. This step was repeated 3 times without delay for each plate. The crystal violet remaining in the biofilms was then eluted with 300 μL of 200 proof ethanol and incubated at room temperature for 45 minutes. To measure the final biofilms, 100 μL of ethanol containing crystal violet was moved to a new microtiter plate. In addition, 10 μL was moved into a second plate containing 90 μL of ethanol to get a 1:10 dilution. Both of these plates were measured on the plate reader. The biofilms quantity was calculated from the 1:10 dilution plate if its read was above background, otherwise it was calculated from the undiluted plate.

The Z factor was determined by running a biofilm assay as above, where half the plate was cultures with positive biofilms and the other half was negative controls. This was done by alternating rows on the 96-well plate to form a checkerboard pattern of positive and negative samples to avoid plate bias and ensure the assay could be carried out without cross contamination of wells.

For the screen, the following procedures were followed. 96-well plates with drugs were thawed for an hour or two on the bench while being kept covered in tinfoil to reduce light exposure. The screen was run as the biofilm assay above in duplicate, however negative and positive controls were placed in the outside columns 1 and 12, and the remaining middle of the microtiter plate was filled with biofilms treated with 10 μM of compounds. The compounds were sourced from Prestwick Chemical Library which contains 1280 compounds that are predominately approved by drug safety agencies such as FDA, EMA, and JAN. The compounds were provided at 10 mM concentration in 100% DMSO. The compounds were further diluted 1:10 in phosphate buffered saline (PBS) and 2 μL of this was used to treat each well.

Validation of the biofilm results and planktonic cell measurements was carried out as above with compounds that were purchased directly from various companies and resuspended in filter sterilized DMSO to 10 mM concentrations. Further dilution of the samples was carried out in sterile DMSO to obtain the appropriate concentrations listed in the graphs.

### Results

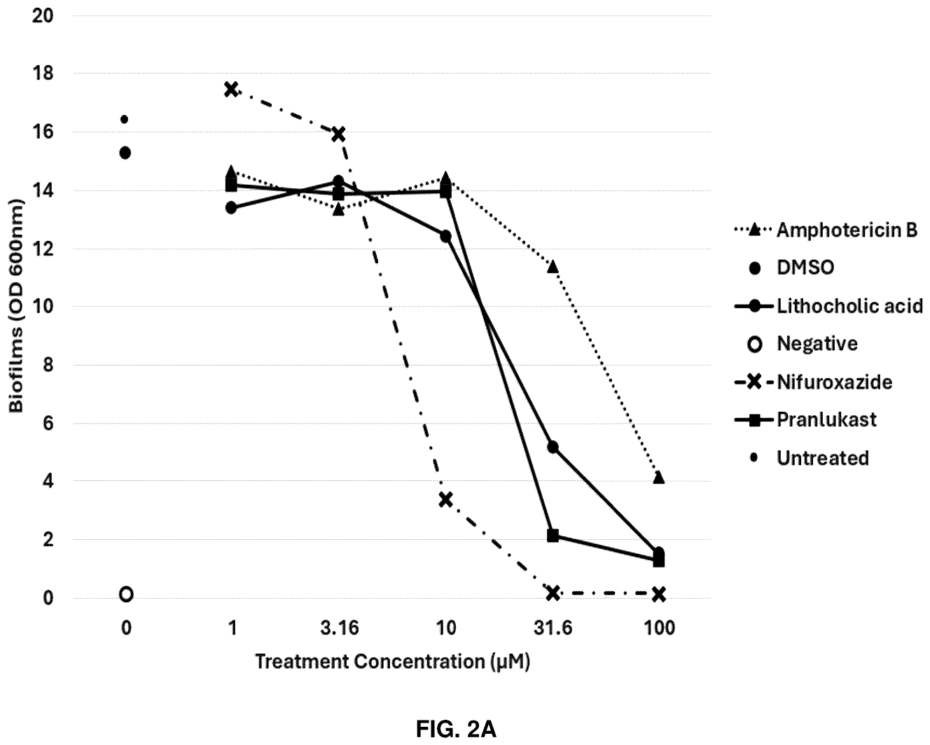

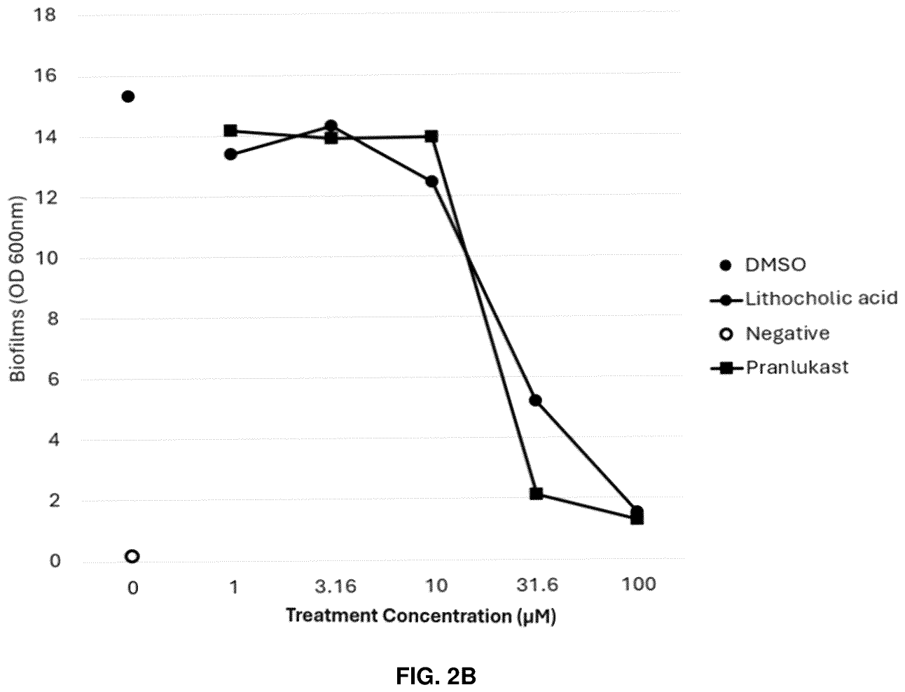

The screen produced the scatter plot shown in FIG. 1. The results of the biofilm assays are shown in FIGS. 2-6. FIGS. 2A-2B show pranlukast reduces biofilms. Pranlukast, DMSO, lithocholic acid, and nifuroxazide all reduce biofilms by the time the compound is at 100 μM, thus making them antibiofilm compounds. Amphotericin B is a compound found in the screen, but is known to act as an antibiofilm compound, and was therefore used as a positive control. Nifuroxazide is another compound found in the screen, but turned out to act as an antibiotic and therefore was removed upon validation of the compounds discovered in the screen.

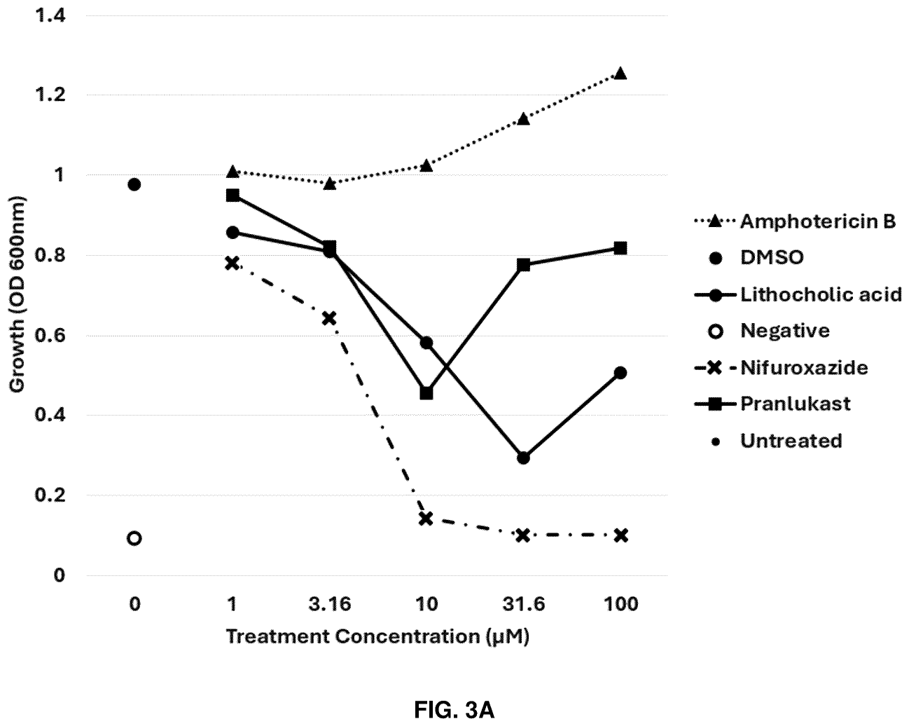

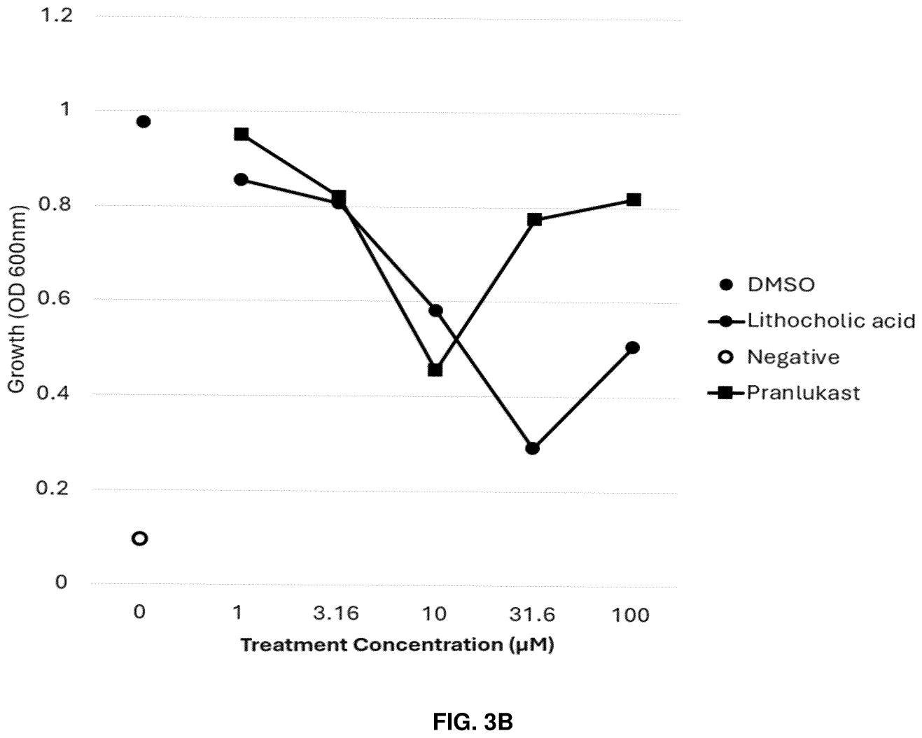

The negative control in FIGS. 3A-3B is illustrated with open circles. As seen in FIG. 3A, the nifuroxazide OD dipped down to the levels of the negative control as the concentration increases, indicating that nifuroxazide acts as an antibiotic and thus not a true antibiofilm compound. In some cases, lithocholic acid and pranlukast OD dipped then rose. This may happen because biofilm dispersion creates complex results when it is read on a spectrophotometer, or it may happen because of precipitation of media components or treatments. If these compounds were killing the cells, one would expect to see the OD simply continue to go down, as seen with nifuroxazide, but the OD for pranlukast and the OD for lithocholic acid rose back up, indicating that the cells are more evenly dispersed and blocking more light.

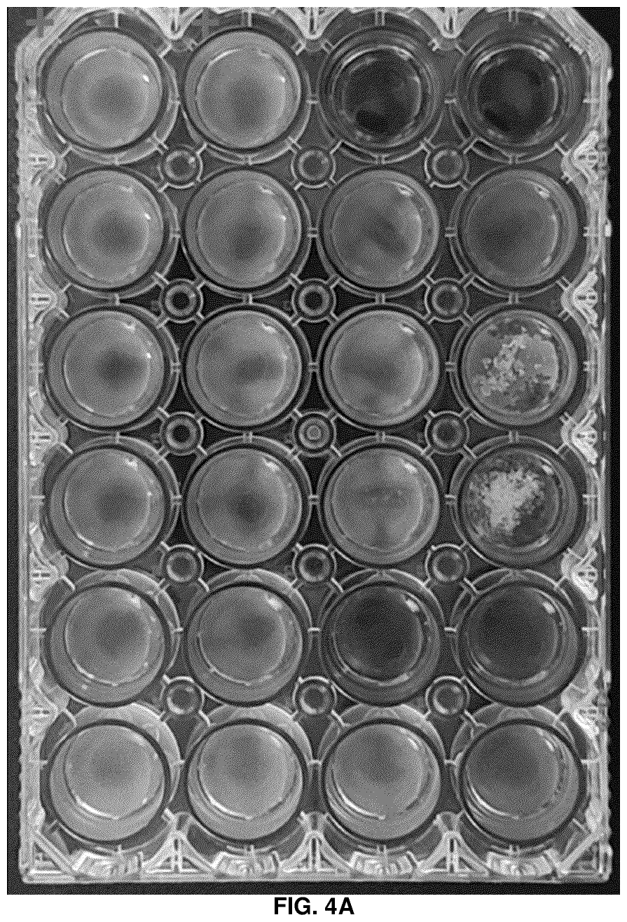

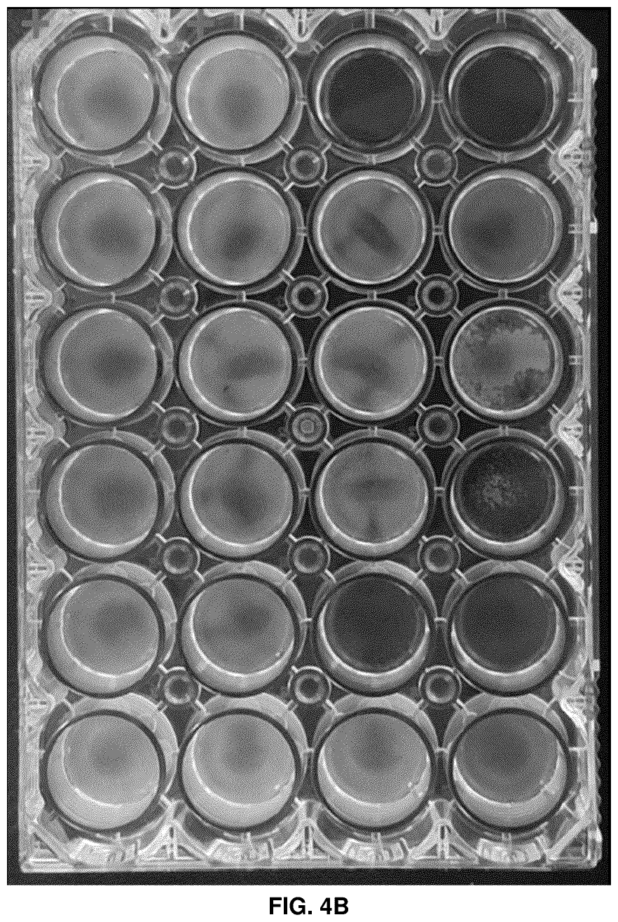

FIGS. 4A-4B show photographs of the biofilm phenotypes following treatment with the compounds. In these photographs, pranlukast is in the row of wells third from the top, and lithocholic acid is in the wells in the row fourth from the top. From left to right, the wells were treated with 3.16 μM, 10 μM, 31.6 μM, and 100 μM of compound.

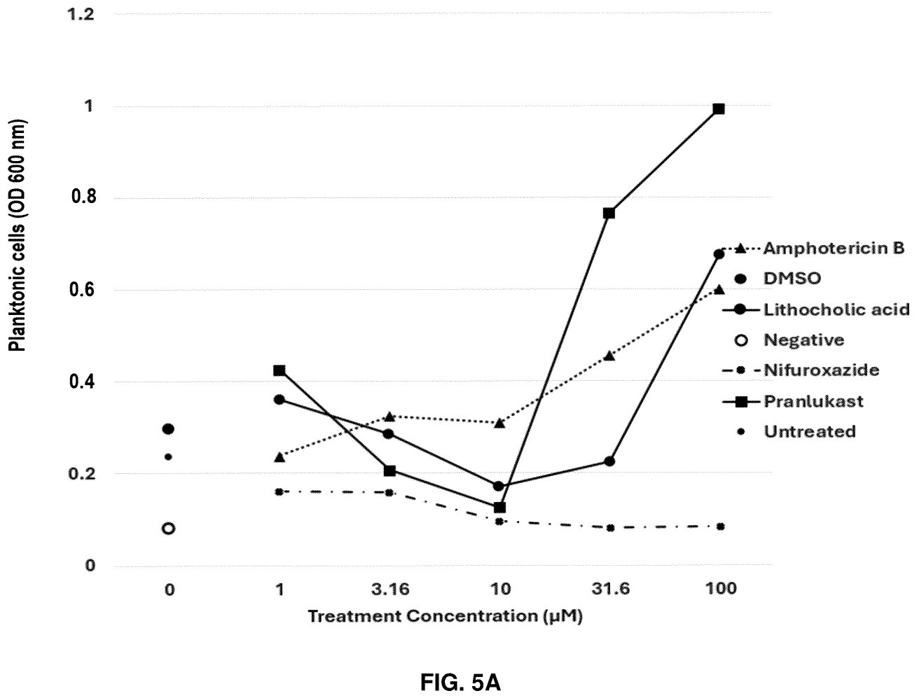

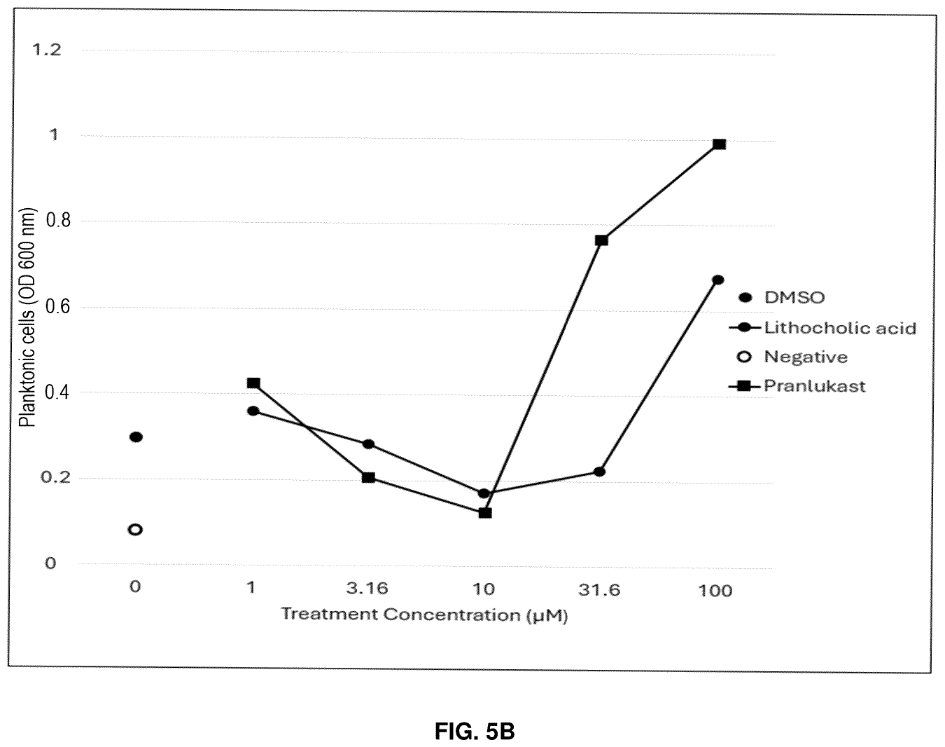

An increase in planktonic bacteria which are not attached to the microtiter plate was evaluated. This is another way of determining whether compounds are true antibiofilm compounds. The cells in the supernatant were measured on a spectrophotometer. FIGS. 5A-5B show the results. In FIG. 5A, the DMSO and untreated controls represent the expected OD of planktonic cells that are still in a biofilm state. As the concentration of a true antibiofilm compound rises, the planktonic cells increase, as is seen with pranlukast, amphotericin B (the positive control), and lithocholic acid. However, if the drug kills bacteria as an antibiotic, then the planktonic cells stay at a low level or drop, as is seen with nifuroxazide in FIG. 5A.

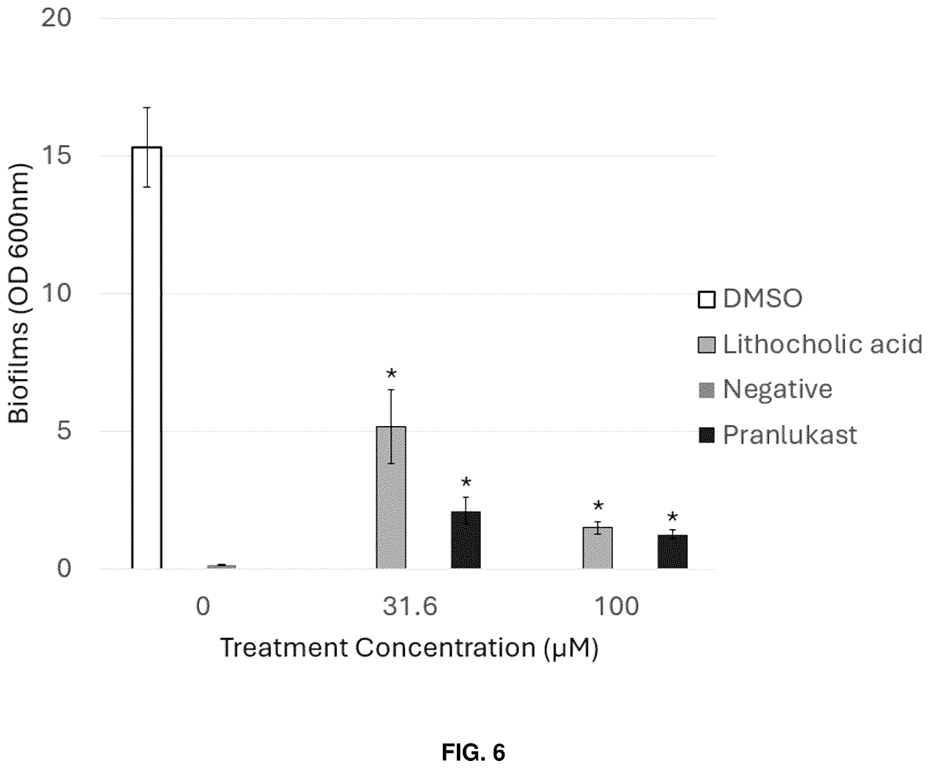

FIG. 6 shows a simple data-reduced example of the true antibiofilm activity of compounds with statistical analysis to determine significance as determined by a T test.

### Pranlukast and Lithocholic Acid Efficacy

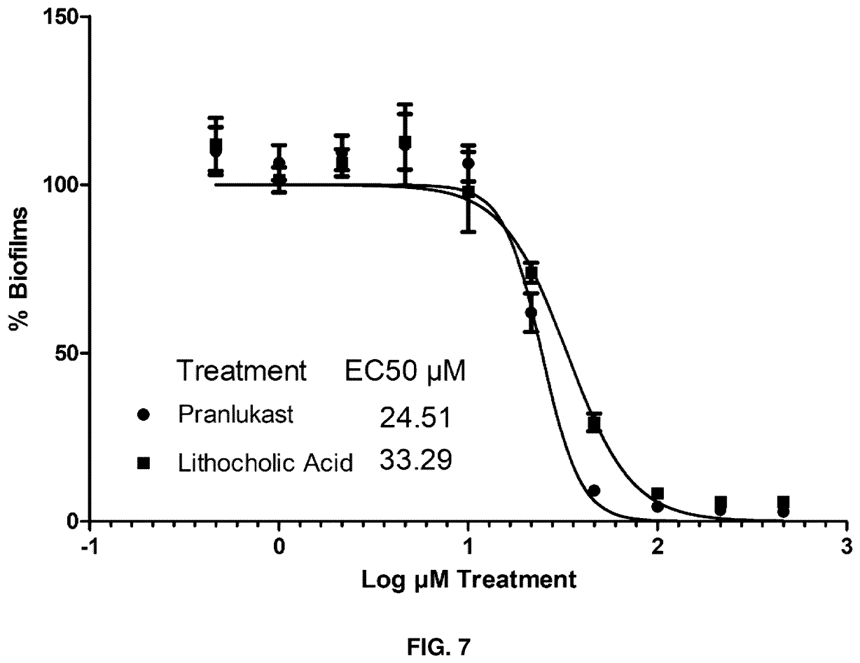

A dose response curve determined the EC₅₀ values of 24.51 μM for pranlukast and 33.29 μM for lithocholic acid. This is shown in FIG. 7. The EC₅₀ curve was fit using PRISM (Graphpad) with a log-dose vs response, three parameters, nonlinear regression.

### Neither Pranlukast Nor Lithocholic Acid Disperse Biofilms

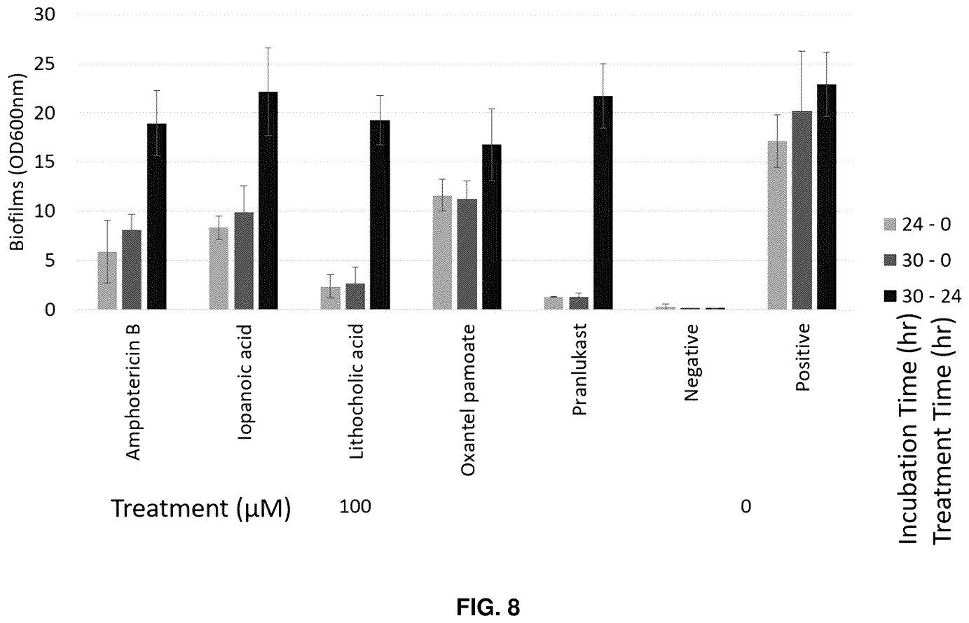

Biofilm dispersal was assayed with treatment of 100 μM of the compounds. Biofilms were formed for 24 hours then treated for an additional 6 hours. If the compound disperses biofilms then the treated black bar should be lower than the light grey in the positive control. The results are shown in FIG. 8, where it is seen that neither pranlukast nor lithocholic acid disperse the biofilms.

### Bacterial Survival

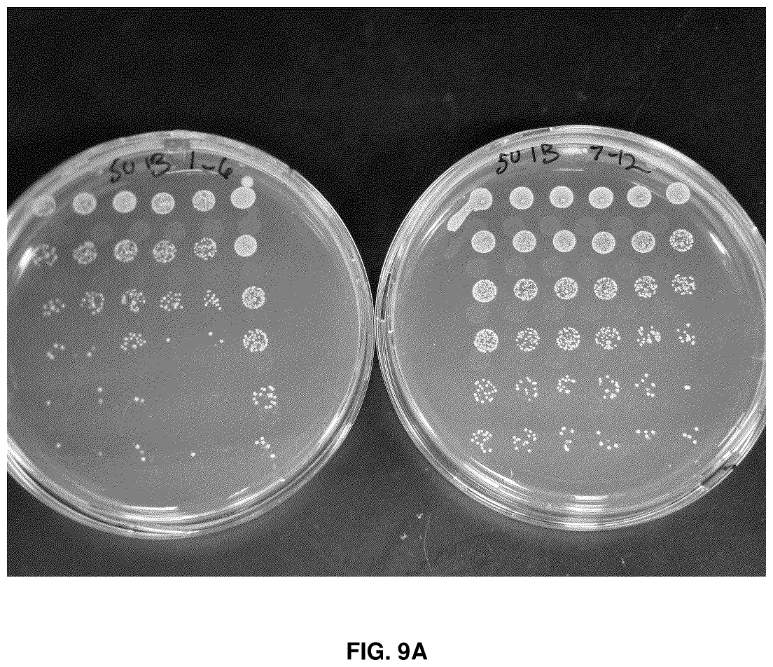

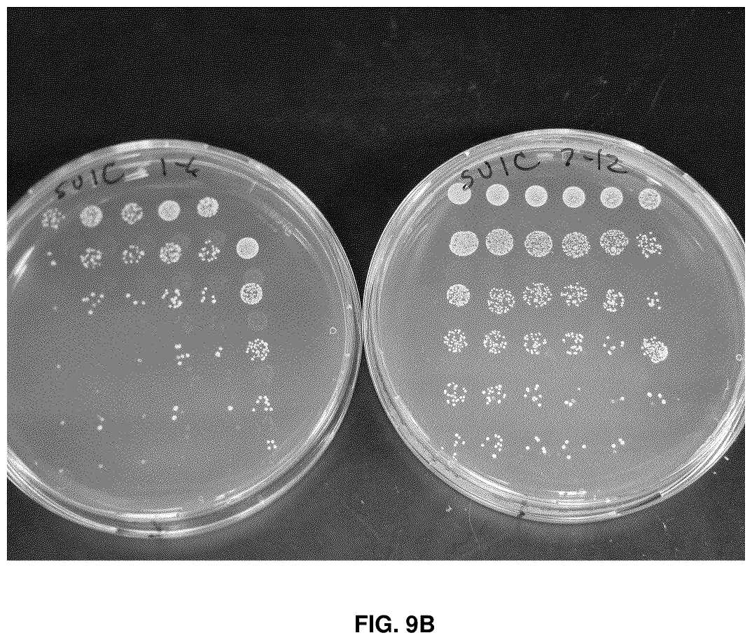

S. mutans was grown in the presence of increasing concentrations of compounds and CFUs were plated to assess bacterial survival. FIGS. 9A-9B show the results. As seen in FIGS. 9A-9B, there were lower CFUs in the presence of the compounds. At no point did the CFU drop to zero indicating that the compounds are not bactericidal. It is believed that the compounds are bacteriostatic.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made, and equivalents may be substituted for elements thereof, without departing from the essential scope of the disclosure. In addition, many modifications may be made to adapt a particular situation or material to the teachings of the disclosure without departing from the essential scope thereof.

What is claimed is:

1. A method of preventing dental cavities in a subject, the method comprising administering an effective amount of an anti-biofilm compound into the mouth of the subject to prevent dental cavities in the subject, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride.

2. The method of claim 1, wherein the anti-biofilm compound is pranlukast.

3. The method of claim 1, wherein the anti-biofilm compound is administered through a mouthwash, toothpaste, a food product, smokeless tobacco, or candy.

4. The method of claim 1, wherein the effective amount is at least about 10 μM.

5. The method of claim 1, wherein the subject is a human.

6. The method of claim 1, wherein the subject is an animal with teeth.

7. The method of claim 1, wherein the subject is a dog, a cat, a cow, a chicken, a turkey, a goat, a pig, a sheep, a horse, a fish, a rabbit, a llama, or a deer.

8. A dental composition comprising:
an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; and
one or more dentally acceptable solvents, flavoring agents, preservatives, fragrances, abrasive agents, surfactants, humectants, thickeners, antimicrobial agents, or enamel-strengthening agents.

9. The dental composition of claim 8, wherein the dental composition is a mouthwash, toothpaste, filling material, sealant, coating, cement, or gel.

10. The dental composition of claim 8, wherein the anti-biofilm compound is pranlukast.

11. The dental composition of claim 8, wherein the effective amount is at least about 10 μM.

12. The dental composition of claim 8, wherein the dental composition is a mouthwash comprising one or more of fluoride, antimicrobial agents, xylitol, alcohol, sodium phosphate, and flavoring agents.

13. The dental composition of claim 8, wherein the dental composition is a toothpaste composition comprising one or more abrasive agents, surfactants, humectants, thickeners, and flavoring agents.

14. The toothpaste composition of claim 13, further comprising a fluoride compound, a calcium phosphate, or a desensitizing agent.

15. A chewing gum composition comprising:
an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; and
a gum base, a sweetening agent, and a flavoring agent.

16. The chewing gum composition of claim 15, wherein the sweetening agent comprises a sugar alcohol, an artificial sweetener, or a natural sweetener.

17. The chewing gum composition of claim 15, comprising one or more of xylitol, sorbitol, mannitol, aspartame, sucralose, and saccharin.

18. The chewing gum composition of claim 15, wherein the effective amount is at least about 0.0003% w/w.

19. The chewing gum composition of claim 15, wherein the anti-biofilm compound is pranlukast.

20. An animal feed composition comprising:
one or more base feed ingredients;
an effective amount of an anti-biofilm compound to inhibit growth of biofilm, wherein the anti-biofilm compound comprises pranlukast, lithocholic acid, oxantel pamoate, amphotericin B, iopanoic acid, nitrofural, nifuroxazide, adamantamine fumarate, or telenzepine dihydrochloride; and
optionally, one or more flavor enhancers, binders, excipients, or nutritional supplements.
